(19) United States
(12) Patent Application Publication
(10) Pub. No.: US 2026/0234520 A1
(43) Pub. Date: Aug. 13, 2026

(54) Apparatus for Culturing a Tissue Section

(71) Applicants: Robert Bosch GmbH, Stuttgart (DE); Robert Bosch Gesellschaft für Medizinische Forschung mbH, Stuttgart (DE)

(72) Inventors: Bernd Scheufele, Koengen (DE); Matthias Schwab, Stuttgart (DE); Walter Aulitzky, Innsbruck (AT); Meng Dong, Stuttgart (DE)

(21) Appl. No.: 19/155,863

(22) PCT Filed: Jan. 18, 2024
(86) PCT No.: PCT/EP2024/051111

§ 371 Date: Aug. 12, 2025

(30) Foreign Application Priority Data
Feb. 14, 2023 (DE) 10 2023 201 202.2

(51) Int. Cl. C12M 3/00; C12M 1/00; C12M 1/26

(52) U.S. Cl. CPC C12M 21/08; C12M 23/38; C12M 23/40; C12M 23/44; C12M 23/48; C12M 29/20; C12M 33/04

(57) ABSTRACT

A device for culturing a tissue section includes a frame with a circumferential profile and a central opening formed by the circumferential profile. The frame has two frame components with a circumferential component profile and a respective central component opening formed by the circumferential component profile. The frame components can be assembled flush with the frame. The device also includes two supporting components that are configured to receive a tissue section. Each frame component has a frame component base body and a frame component insert that can be assembled to the frame component. For each frame component, the frame component insert and the frame component base body are configured to retain the supporting component such that the supporting component spans the opening on the frame component.

The present invention relates to a device for culturing a tissue section, in particular a tumor tissue section, for example for drug testing. In addition, the present invention relates to a culturing system and method for culturing a tissue section.

### PRIOR ART

Unfortunately, not all cancer patients with solid tumors respond well to drug-based cancer therapy. Some significant reasons for this are the individual variability and heterogeneity of the tumors as well as the complexity of the tumor microenvironment. There is a pressing need for increasingly personalized cancer treatment that takes into account the cellular characteristics of individual patients in order to derive an individually optimized therapy. Furthermore, there is a high need for a reliable in vitro tumor model that sufficiently reflects the in vivo situation, and that may be used for the development and preclinical testing of new antitumor agents.

As part of a biopsy or surgery, cancer tissue is taken from a patient. The cancer tissue is then cultured and used for in vitro testing of anti-cancer drugs and for studying the individual drug effects. For in vitro testing, a suitable cell-based tumor model is required that may be obtained from tissue samples from cancer patients and that depicts key aspects of the complex in vivo tumor biology. From the tumor sample taken, thin tissue sections with a typical tissue section thicknesses of approximately 150 μm to 400 μm are produced. These tumor tissue sections are particularly suitable for use as a tumor model because they maintain in vivo tissue heterogeneity with various cell types and the preserved tumor microenvironment.

Various methods have been developed for culturing tumor tissue sections, but they allow for limited culturing time. After a few days, the viability of the tissue section cells decreases significantly and the cells die. The culturing time is very limited because, when the tumor is removed, it is inevitable that the blood supply will be fully blocked and supplying physiological concentrations of oxygen to the ex vivo tumor tissue cultured in medium is very difficult. This is due to the different oxygen absorption capacity in culture medium as compared to blood.

According to the current prior art, the viable tumor tissue sections are cultured floating in a suitable medium or lying on a filter. The most commonly used culturing system is a tissue culture insert for multiwell plates (Millipore filters). With this system, the tissue sections are cultured at the air-liquid interface, thereby achieving a higher supply of oxygen. It is true that the known systems with tissue culturing devices at the air-liquid interface may partially address this deficiency by exposing a tissue side to air, whereby this type of tissue section culturing is clearly superior to the liquid culture, but this leads to the formation of an artificial gradient, which is oriented from the air side to the medium side, in particular such that only the upper, the air-facing cell layers correspond to the original tissue from which the tumor sections were obtained in their morphology and the properties, whereas the cell layers facing away from the air have signs of hypoxia and necrosis.

A culturing approach is known from WO 2019/029947 A1 in which a fluid supply of culture medium to the tissue sections is realized instead of static culturing. A so-called Perfusion Air Culture (PAC) system with adjustable medium or drug supply is used for this purpose. The device comprises a frame, at least one supporting component, wherein the supporting component is configured to receive a tissue section and at least one liquid absorbent strip element. The frame is configured to hold the at least one supporting component and the at least one strip element.

### DISCLOSURE OF THE INVENTION

A tissue section culturing device is proposed. In particular, the tissue section is a tumor tissue section. The device comprises a frame having a circumferential profile and a central open space formed by the circumferential profile. The frame comprises two frame components, each with a circumferential component profile and a central component opening formed by the circumferential component profile. Both frame components are preferably couplable to the frame in such a way that they cover it, by means of a connecting element. Each frame component further comprises a frame component base body and a frame component insert that may be connected to the frame component. Preferably, the profile of the frame component insert comprises a collar that may be inserted into the opening of the frame component base body. In addition, the device comprises two supporting components, one supporting component for each frame component. The two supporting components are in particular organotypic and configured to receive a tissue section. For each frame component, the frame component insert and the frame component base body are configured to hold a supporting component when assembled. For this purpose, the supporting component may be clamped by the frame component insert and the frame component base body. Each frame component is configured to hold a supporting component after assembly so that it is clamped to the frame component spanning the opening of the frame component. In this way, the frame component insert and the frame component base body may hold the supporting component circumferentially on all sides equally. Alternatively, it may also be provided that the supporting component is not held on all sides but, for example, on two opposite sides. The other sides of the supporting component may then, for example, be drawn through the opening of the frame component base body. The profile of the frame component insert may comprise cuts that may be used to better position the supporting component.

For each frame component, a supporting component is interposed between the frame component base body and the frame component insert. The supporting component is clamped between the frame component base body and the frame component insert inserted into it by mating them together. This connection may be separated by separating the frame component base body and the frame component insert from one another. The supporting component may then be removed and transported elsewhere, for example for further examinations of the tissue section. A new supporting component may then be easily re-inserted and clamped as described above.

The two frame components are then assembled to the frame. The two frame component base bodies are preferably aligned with one other for this purpose. After the two frame components are assembled to the frame, the tissue to be examined or to be cultured, for example a thin tissue section, and in particular a tumor tissue section, may be held between the two supporting components. The two supporting components together may form a chamber in which the tissue section may be inserted prior to assembly of the frame, so that it is held on both sides by the supporting components. This provides optimal oxygen concentrations to the tissue section on both sides, thereby allowing for atmospheric culturing of the tissue section.

The supporting components may be directly supplied with fluid. The fluid may be admitted between the supporting components, in particular in the chamber formed by the supporting components. This allows for constant perfusion with a culturing medium having a desired concentration of nutrients. In addition, a solution of active ingredients, for example a medication solution, may be introduced. The result is a supply of nutrients and, if necessary, a uniform application of medications. Thus filter papers, as are used for supplying in current systems, may be dispensed with. The device thus allows for the testing of active ingredients and medications while maintaining the native individual tissue, for example a tumor tissue, under largely physiological conditions. The total amount of perfusate and thus the consumption of the active ingredient to be tested may also be kept low.

In the present case, a “circular profile” and a “center opening formed by the circumferential profile” are understood to mean any enclosure that is used to hold or stabilize one or more supporting components clamped or received in the (circumferential) frame.

The frame, or the two frame components, is/are preferably generally substantially rectangular or narrowly cuboid and correspondingly has/have four sides.

Preferably, the at least one supporting component is formed from a material selected from the group consisting of cotton fabric, nylon fabric, polycarbonate, cellulose hydrogel, animal intestine, in particular porcine intestine, 3D-printed biomaterials, in particular 3D-printed alginate fiber or 3D-printed collagen fabric. Preferably, the at least one supporting component is or comprises a grid-shaped and oxygen permeable fabric.

The frame and components thereof may preferably be formed from a solvent-resistant material. In particular, biocompatible polymers such as polycarbonate (PC), polypropylene (PP), acrylonitrile butadiene styrene copolymer (ABS), polyethylene terephthalate (PET), polylactic acid (PLA), polyether ketone (PEEK), and polyoxymethylene (POM), which may be easily cleaned and/or sterilized, may be used as the material. For example, 3D-printing or injection molding may be used to manufacture the components. Alternatively, other materials, such as metals or ceramics and corresponding manufacturing methods may be used.

Preferably, a connecting element is provided by means of which the frame components may be assembled to the frame. The connecting element is preferably formed on a frame component insert. When assembling the frame, the connecting element may be connected to the other frame component insert. The latter may additionally comprise a corresponding connection feature with which the connecting element interacts in the connection, for example an indentation, a hook, an eye, or the like. As described above, the frame component inserts in the assembled device are preferably externally disposed in each frame component. A secure and easily accessible connection concept that ultimately holds the whole device together is thereby realized. The connecting element is preferably configured as a click-fit connection, a guide rail, a clamp connection or a fastener.

Advantageously, the frame comprises a guide for supplying fluids. This guide is preferably formed by connecting or assembling the two frame components. Particularly preferably, the guide is formed to supply fluid through the connection or assembly of the two frame component base bodies. Alternatively, the guide is located on one of the two frame components. A liquid, for example a culturing medium or a solution of the active ingredient, in particular a medication solution of the device—and thus the tissue section to be cultured in the device—may be fed through the guide and thus the tissue may be perfused with the fluid. The fluid is thereby introduced into the interim opening within the frame, preferably between the supporting components, where the fluid comes into contact with the fabric held therein.

The fluid supply guide is preferably divided into a plurality of different sections within the frame profile. An outer section may be flared so that a conventional tube-shaped injection cannula may be easily inserted. In a central section, a constriction with a diameter of, for example, 0.3 mm to 1.2 mm may be provided, with which the cannula is particularly held straight and rigid. An inner section may in turn be flared and form a cavity. The cavity is connected to the two supporting components and improves the fluid transition of the fluid from the small cannula opening to the flat and closely spaced supporting components. The cavity may be filled with a fluid for this purpose, for example with the culturing medium and/or the medication solution. As a result, a complete cannula connector is formed. A conventional cannula may be inserted into the guide up to the chamber between the supporting components. The cannula may in turn be operated via a hose with a pumping mechanism, such as a syringe pump or a peristaltic pump. The cannula may be used to precisely supply the tissue section with the culturing medium and, if necessary, with the medication solution-in particular with a flow rate in the range of μl/h to ml/h.

Advantageously, the frame component base body has a trapezoidal protrusion on an outward side. The trapezoidal protrusion extends the profile of the frame component base body in the plane. The narrow side of the trapezoidal protrusion faces outward and the long side faces toward the central component opening. The trapezoidal protrusion thus widens towards the central component opening. Preferably, the associated supporting component on this side is also trapezoidal in shape and adapted to the protrusion. In the assembled state, the supporting component with the trapezoidal side rests on the frame component base body on the side facing away from the component insert. The side of the supporting component may be drawn through the opening of the frame component base body for this purpose. Advantageously, the above described guide for fluid supply is arranged in the trapezoidal protrusion. Due to the trapezoidal shape, the externally supplied fluid may better distribute inwardly and a homogeneous flow of fluid is produced into the preferably rectangular opening and to the fabric section. Thus filter papers, as are used for supplying in current systems, may be dispensed with.

To improve the distribution of the liquid flow even more, the frame component base body may have structures at the surface of the trapezoidal protrusion to distribute the liquid. The structures are, for example, protrusions that protrude from the frame component base body. The structures abut or penetrate the supporting component. Thus, the structures present obstacles to the fluid flow through which the fluid flow is deflected and widened. The structures may take any shape in their cross-section, for example round, rectangular or oval, and preferably have a cross-sectional size of between 0.1 mm and 1.0 mm as well as a height corresponding to the distance between the frame and component base body. The structures may be arranged regularly or irregularly and may be arranged in suitable numbers and distances. In addition, elongated fluidic barriers may be provided in the trapezoidal protrusion, particularly at its edges, to prevent leakage of the fluid in the trapezoidal protrusion.

In addition, a culturing system for the culturing of tissue sections, in particular for the testing of active ingredients and/or medications, is proposed. The culturing system comprises a device according to the invention as described above, a retaining frame and a receiving vessel. The device is pushed into and held by the retaining frame. The retaining frame may preferably have a holding and positioning structure on both sides with a lower stop. The retaining structure may also prevent unwanted opening of the frame, frame components, and/or chamber. In this case, the connecting element described above may be omitted on the frame. The retaining frame is then inserted into the receiving vessel. The holding frame is configured to be insertable into the receiving vessel and may preferably be placed in a predetermined depth. This has the advantage that, for example, a conventional vessel, in particular a ventable tube, for example a correspondingly sized centrifuge tube, may be used as a receiving vessel in which the device according to the invention may be fastened using the retaining frame. The receiving vessel may comprise a lid, in particular a screw cap, which allows for sterile gas exchange as well as access to the guide for fluid supply, for example through a septum slot. An injection cannula for guiding the fluid supply may be inserted through the access. Additionally or alternatively, the access allows a removal cannula to be inserted into the receiving vessel. The sealability of the tube with the lid is not affected. The retaining frame may have a structure for holding and guiding the cannula, in particular the removal cannula.

The tube may be selected according to a preferred embodiment from a tube holding between 10 and 100 ml, and in particular a 10, 15, 20, 30, 40, 50, 60, 70, 80, 90, or 100 ml tube. Preferred dimensions for the frame range from 18 mm to 28 mm×21 mm to 31 mm, in particular 23×26 mm, as well as for the opening 9 mm to 19 mm×10 mm to 20 mm, in particular 14×15 mm.

With this system according to the invention, a vertical orientation of the device according to the invention is advantageously achieved in the receiving vessel and thus also a vertical orientation of the tissue section in a closed, sterile-ventilated receiving vessel. The system may then be kept in an incubator under a controlled atmosphere, for example. With a vertical orientation, the device is preferably also accommodated in the receiving vessel such that there is a guide on the top side of the frame for fluid supply, for example by means of a perfusion needle, which is connected to a perfusion tube, if necessary. This may preferably be connected to a syringe and under control of a pump mechanism, e.g. a syringe pump or a peristaltic pump, via which a controlled perfusion is possible. The fluid introduced via this system may be collected in the receiving vessel after passing through the supporting component and thus also the tissue at the bottom of the receiving vessel.

In addition, a method of culturing a tissue section is proposed. This method utilizes the above-described device or above-described culturing system.

The frame components are initially prepared. Two frame component base bodies and two frame component inserts, one for each frame component, as well as two supporting components are provided. One supporting component is placed on each frame component insert, namely, such that the supporting component spans the component opening and extends into the profile of the frame component insert. In this case, all sides may extend circumferentially equally into the frame component-insert profile. Alternatively, sides of the supporting component may also be left free. The frame component insert is then inserted into the frame component base body and thus a frame component is formed. The supporting component is now retained by the sides that have extended into the frame component-insert profile and are covered by the frame component base body profile. It may be provided to pull sides of the supporting component through the opening of the frame component base body to the other side. In this way, the retaining effect and leakage safety may be improved and the sides may be used for additional functions, such as a better distribution of the supplied fluid. The frame components may then be sterilized.

A fabric section is placed on a supporting component in one of the frame components. The other frame component is then placed on top, wherein the two frame component base bodies and the supporting component of the other frame component rests on the fabric section. The supporting components thus form a chamber in which the tissue section is stored. The frame components are then connected with one another to the frame. Preferably, a connecting element, which is preferably arranged on one of the frame component inserts, is connected to the other frame insert component for this purpose. In so doing, the tissue section is clamped and held between the supporting components.

Preferably, the frame is pushed into a retaining frame with the fabric section. The retaining frame together with the frame is then pushed into a receiving vessel, for example a ventable tube. The receiving vessel may then be sealed with a lid. In addition, the receiving vessel may be placed in an incubator.

For culturing, the culturing system may be fluidly connected to a supply system that provides a culturing medium and to a collection system that collects the fluid from the culturing system. A fluid, in particular a culturing medium, is fed in the opening surrounded by the supporting components within the frame, i.e. the chamber formed by the supporting components. A defined flow rate and/or a desired time period may be set for the feed. The fluid may penetrate the supporting components and, in particular, act on both sides of the tissue section in the receiving vessel. Metabolic products pass from the tissue section into the fluid. The fluid with the metabolic products may then be dissipated, particularly outside the frame.

Guides for cannulas may be provided in the lid for the supply and discharge of the fluids. To supply fluids, an injection cannula is passed through the lid to the frame. If there is a fluid supply guide in the frame, the injection cannula may be inserted into it. To remove fluids, an extraction cannula may additionally be inserted into the receiving vessel through the lid, wherein it is preferably inserted outside the frame and advantageously reaches to the bottom of the receiving vessel in order to remove the liquid collected therein.

For drug testing and analysis, the supply system with which the culturing system is associated may additionally provide a medication solution that is supplied within the frame into the opening surrounded by the supporting components. A defined dose and a desired time period may be set for the supply of the medication solution. The medication solution may penetrate the supporting components and in particular act on both sides of the tissue section in the receiving vessel. In addition, the collection system may comprise or be fluidly linked to an analysis system that performs an analysis of the metabolic products. At the end of medication testing, the frame may be removed from the receiving vessel. The frame components may be disconnected from one another and the tissue section may be removed for further examination.

### BRIEF DESCRIPTION OF THE DRAWINGS

Exemplary embodiments of the invention are shown in the drawings and explained in more detail in the following description.

FIGS. 1A-H show isometric views of basic components of the device according to the invention.

FIGS. 2A, B show isometric views of the device according to the invention before (FIG. 2A) and after assembly (FIG. 2B).

FIG. 3A shows a side view of the device according to the invention of FIG. 2 with a cannula.

FIG. 3B shows an enlarged and 90° rotated cross-sectional view of an excerpt of FIG. 3A.

FIG. 4A shows a top plan view of a frame component base body with a supporting component according to a further embodiment.

FIG. 4B-D show three enlarged excerpts from FIG. 4A.

FIGS. 5A-D show isometric representations and a top plan view (FIG. 5C) of basic components of the culturing system according to the invention.

FIG. 6 shows an isometric view of the culturing system according to the invention.

FIG. 7 shows a flowchart of an exemplary embodiment of the culturing method according to the invention.

FIG. 8 shows a flowchart of a method for medication testing and analysis.

### EXEMPLARY EMBODIMENTS OF THE INVENTION

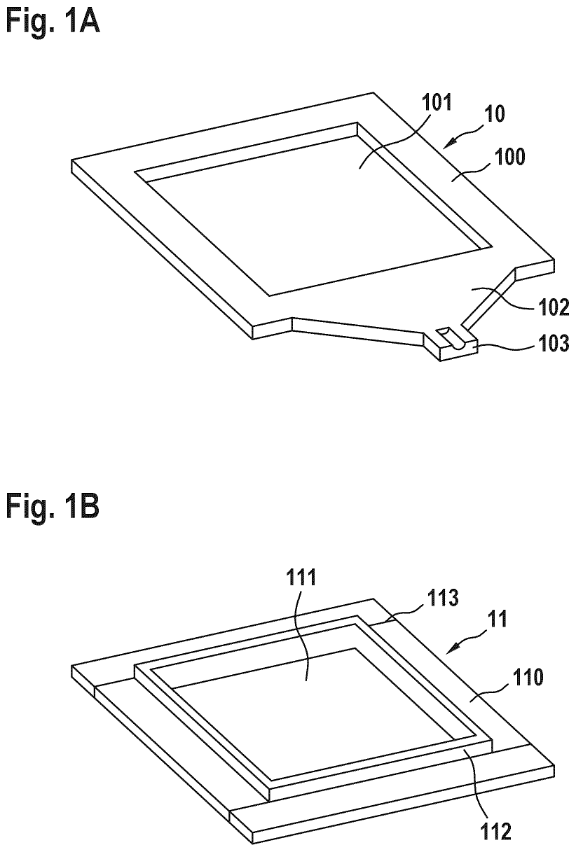

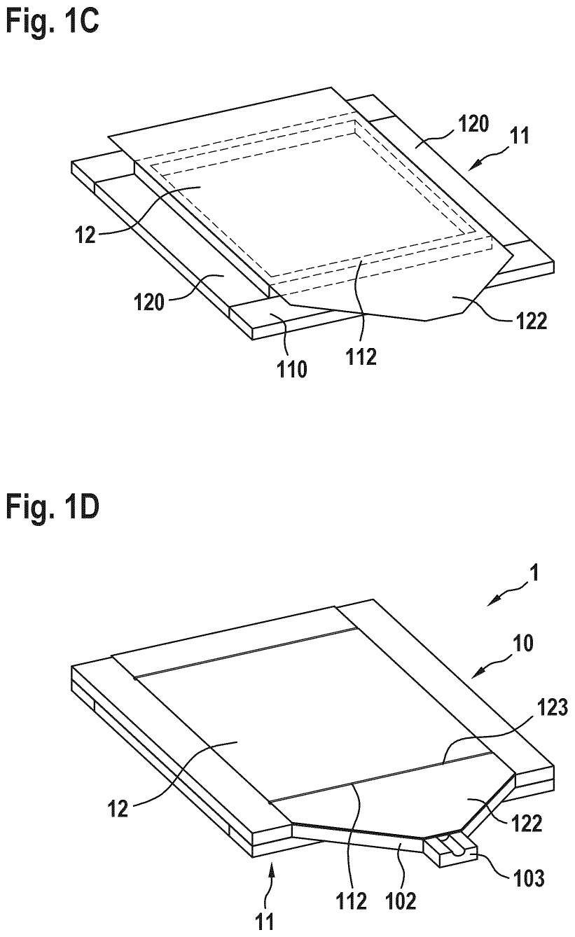

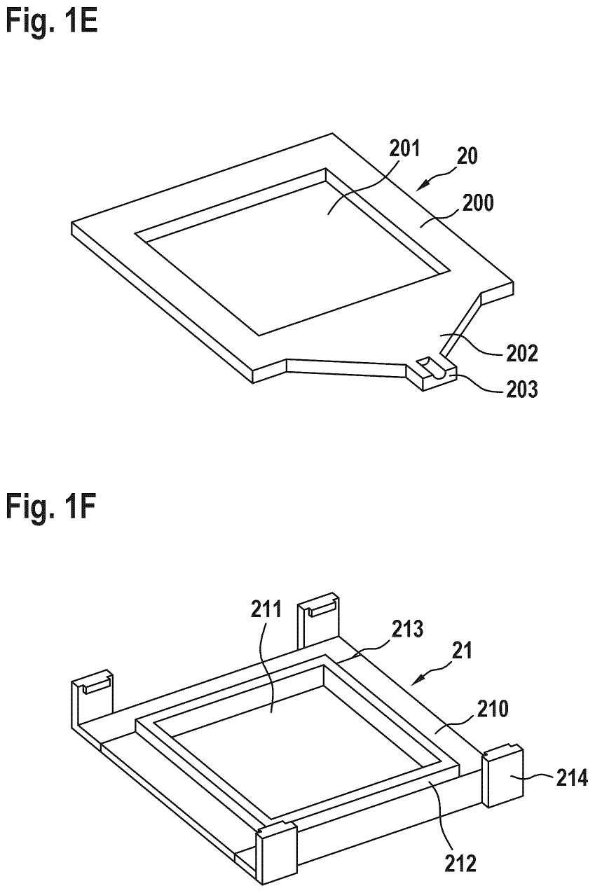

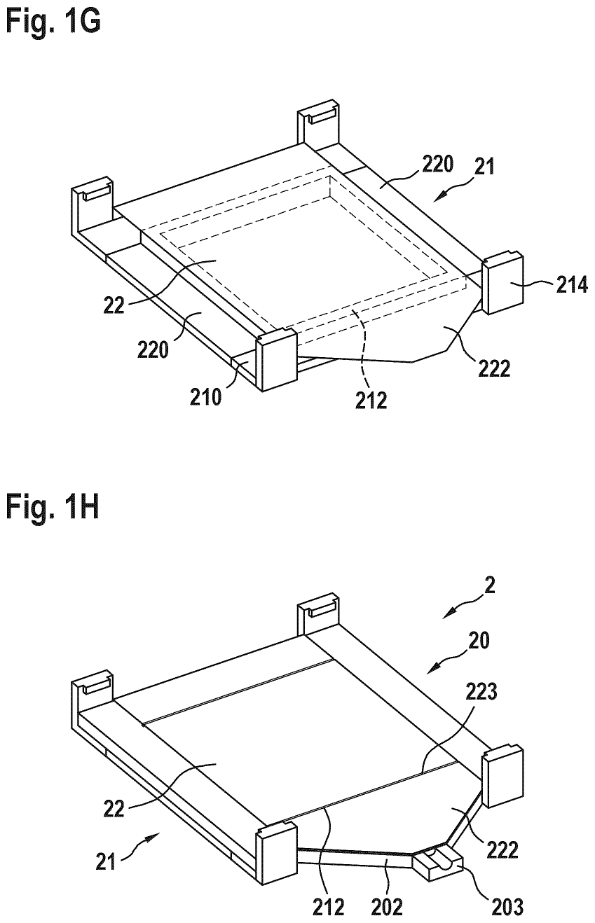

FIGS. 1A-H show the basic components of the device according to the invention. FIGS. 1A-D show a first frame component base body 10 and a first frame component insert 11, which are assembled into a first frame component 1 as shown in FIG. 1D, as well as a first organotypical supporting component 12. The first frame component base body 10 shown in FIG. 1A comprises a profile 100 that surrounds a central opening 101. The frame component base body profile 100 and the opening 101 are square, with the exception of one side where the profile 100 has a trapezoidal protrusion 102 in the plane. The trapezoidal protrusion 102 is isosceles, the short side faces outward and the long side transitions into the remaining profile 100 such that the trapezoidal protrusion 102 flares inwardly. One half 103 of a guide, or in other words, one cannula connector 33 is arranged on the short side, which will be discussed in detail below. The first frame component insert 11 shown in FIG. 1B also comprises a profile 110 that is rectangular and has the same dimensions as the profile 100 of the associated first frame component base body 10. The frame component insert profile 110 surrounds a likewise rectangular, centrally disposed opening 111. At the edge of the frame component insert profile 110, a collar 112 projecting perpendicular to the profile 110 is formed, which joins the profile 110 inwardly. In the frame component insert profile 110, cuts 113 are machined on two opposite sides which serve to position the first supporting component 12.

In FIG. 1C, the first supporting component 12 is placed on the first frame component insert 11. For example, the supporting component 12 here is a grid-shaped and oxygen permeable fabric made of cotton. One side 122 of the supporting component 12 is also trapezoidal in shape and corresponds in shape and dimensions to the trapezoidal protrusion 102 of the frame component base body 10. Two other sides 120 of the supporting component 12 are retained in the cuts 113 of the profile 110.

In FIG. 1D, the first frame component base body 10 is placed on top of the first frame component insert 11. The first frame component base body 10, the first frame component insert 11 and the first supporting component 12 are aligned with each other, the profile 100 of the first frame component base body 10 lies flush on top of the profile 110 of the first frame component insert 11, the collar 112 of the first frame component insert 11 engages with the opening 101 of the first frame component base body 10 and the trapezoidal side 122 rests on the trapezoidal protrusion 102 of the frame component base body 10. For this purpose, the trapezoidal side 122 is guided upwards from below through the opening 101 of the frame profile base body 10 and then folded outwards along the line 123. The opposite side of the first supporting component 12 is designed in the same manner. The first supporting component 12 is retained at the sides 120 of the first frame component base body 10 and the first frame component insert 11. Thus, the first frame component 1 is formed.

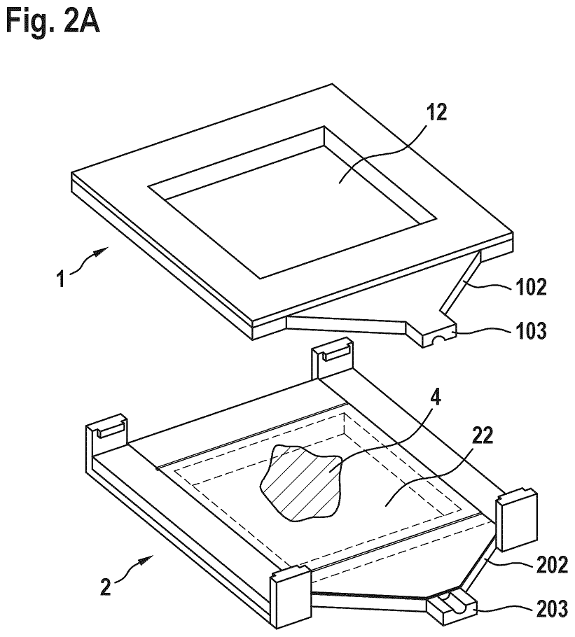

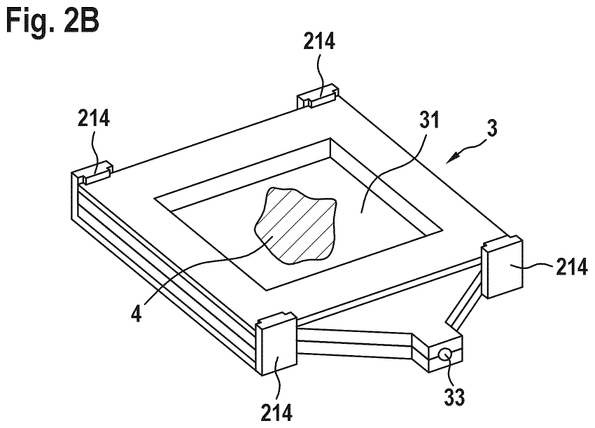

FIGS. 1E-H show a second frame component base body 20 and a second frame component insert 21, which are assembled into a second frame component 2 as shown in FIG. 1H, as well as a second organotypical supporting component 22. The second frame component base body 20 shown in FIG. 1E is formed with the same shape and dimensions as the first frame component base body 10. The second frame component base body 20 has a profile 200 that surrounds a central opening 201. The frame component base body profile 200 and the opening 201 are square except for one side where the profile 200 comprises a trapezoidal protrusion 202 in the plane. The trapezoidal protrusion 202 is isosceles, the short side faces outward and the long side transitions into the remaining profile 200 such that the trapezoidal protrusion 202 flares inwardly. The other half 203 of the cannula connector 33 is disposed, which will be discussed in detail below, on the short side. The second frame component insert 21 shown in FIG. 1F also has a profile 210 that is rectangular and has the same dimensions as the profile 200 of the associated second frame component base body 20. The frame component insert profile 210 surrounds a likewise rectangular, centrally disposed opening 211. At the edge of the frame component insert profile 210, a collar 212 projecting perpendicular to the profile 210 is formed, which joins the profile 210 inwardly. In the frame component insert profile 210, cuts 213 are machined on two opposite sides which serve to position the second supporting component 22. In contrast to the first frame component insert 11, the second frame component insert 21 comprises four connecting elements 214 disposed outside at the corners of the frame component-insert profile 210 in the form of click elements. Their function is described in connection with FIG. 2.

In FIG. 1G, the second supporting component 22 is placed on the second frame component insert 21. The supporting component 22 here is also a grid-shaped and oxygen permeable cotton fabric. One side 222 of the supporting component 22 is also trapezoidal in shape and corresponds in shape and dimensions to the trapezoidal protrusion 202 of the frame component base body 20. Two other sides 220 of the supporting component 22 are retained in the cuts 213 of the profile 210. In FIG. 1H, the second frame component base body 20 is placed on top of the second frame component insert 21. The second frame component base body 20, the second frame component insert 21 and the second supporting component 22 are aligned with each other, the profile 200 of the second frame component base body 20 lies flush on top of the profile 210 of the second frame component insert 21, the collar 212 of the second frame component insert 21 engages with the opening 201 of the first frame component base body 20 and the trapezoidal side 222 rests on the trapezoidal protrusion 202 of the frame component base body 20. For this purpose, the trapezoidal side 222 is guided upwards from below through the opening 201 of the frame profile base body 20 and then folded outwards along the line 223. The opposite side of the second supporting component 22 is designed in the same manner. The second supporting component 22 is retained at the sides 220 of the second frame component base body 20 and the second frame component insert 21. Thus, the second frame component 2 is formed.

FIGS. 2A, B show the assembly of the two frame components 1, 2 from FIG. 1 to a frame 3 in which a fabric section 4 is received between the supporting components 12 and 22. FIG. 2A shows the frame components 1, 2 and the fabric section 4 separately. The first frame component 1 is rotated with the first frame component base body 10 towards the second frame component base body 20 of the second frame component 2. The first frame component 1 and the second frame component 2 are aligned such that the profiles 100 and 200, the openings 101 and 201 as well as the trapezoidal protrusions 102 and 202 of the base bodies 10, 20 each lie one above the other. The fabric section 4 is placed on the supporting component 22 of the second frame component 2 within the opening 22. The first frame component 1 is then placed on top of the second frame component 2. In FIG. 2B, the frame 3 is shown in the assembled state. The frame components 1, 2 surround a central opening 31, which corresponds to openings 111 and 211. The fabric section 4 is disposed between the base bodies 10, 20 of the first frame component 1 and the second frame component 2 and within the opening 31, and is located in a chamber formed by the two supporting components 12 and 22 in the openings 101, 111, 201, 211. The connecting elements 214 of the second frame component insert 21 enclose the second frame component 2 and the first frame component 1 and are clipped into the first frame component insert 21 of the first frame component 1. The first frame component insert 21 may have corresponding recesses for this purpose, which are not shown. Alternatively, other types of connecting elements 214 may be selected. In this way, a rigid connection is realized and the fabric section 214 is firmly and securely clamped between the supporting components 21 and 22. The connection is releasable by disengaging the connecting elements 214. The two cannula connector halves 103, 203 lie on top of one other and together form the cannula connector 33, which is described below.

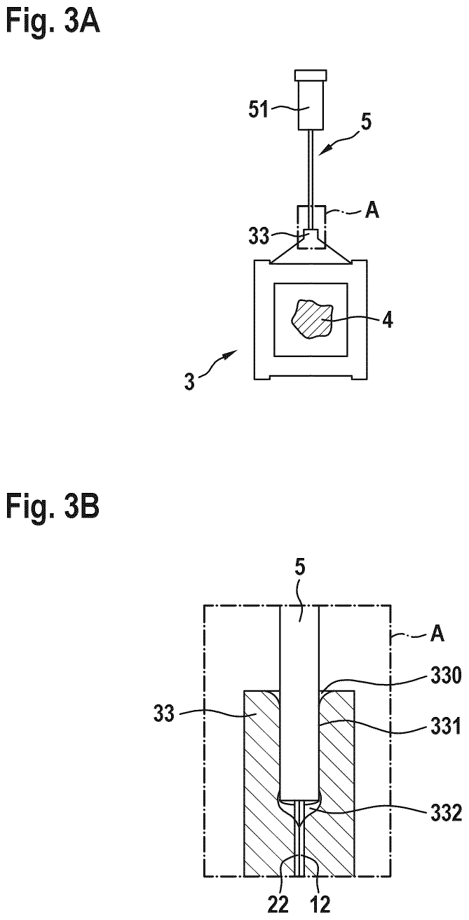

In FIG. 3A, the device according to the invention is shown in an upright position, such that the cannula connector 33 points upward. An injection cannula 5 is inserted into the cannula connector 33. The latter comprises a Luer connector 51 for fluidic connection to a supply system on the opposite side. In FIG. 3B, an enlarged sectional view of excerpt A of FIG. 3A is shown, rotated by 90° as compared to FIG. 3A. The cannula connector 33 has a widening 330 in the upper region, which functions as an injection aid. A narrower cannula channel, also described as a constriction 331, is provided in the middle region with respect to the widening 330, which encloses the cannula 5 and thereby holds it securely. The lower region expands to a cavity 332 in which the fluid to be introduced is held. From the cavity 332, the fluid admitted through the cannula 5 between the two supporting components 12, 22 can penetrate into the chamber and thus towards the tissue section 4.

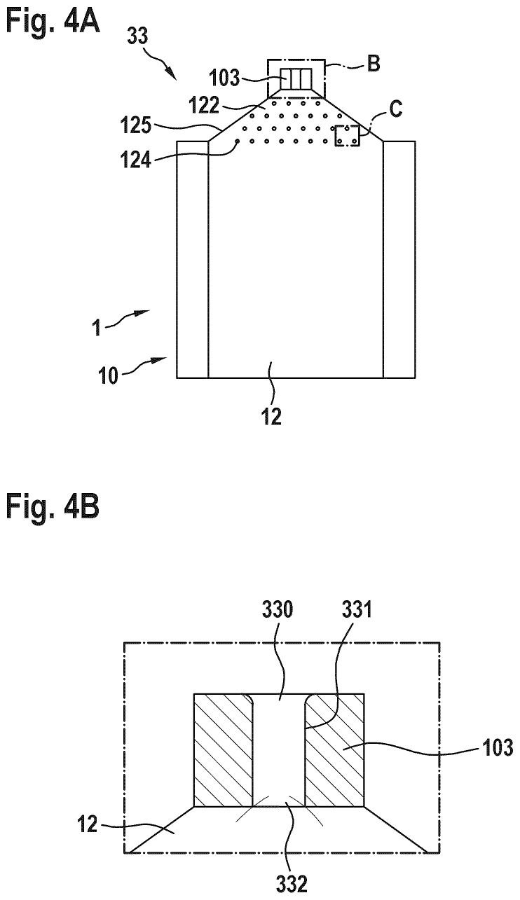

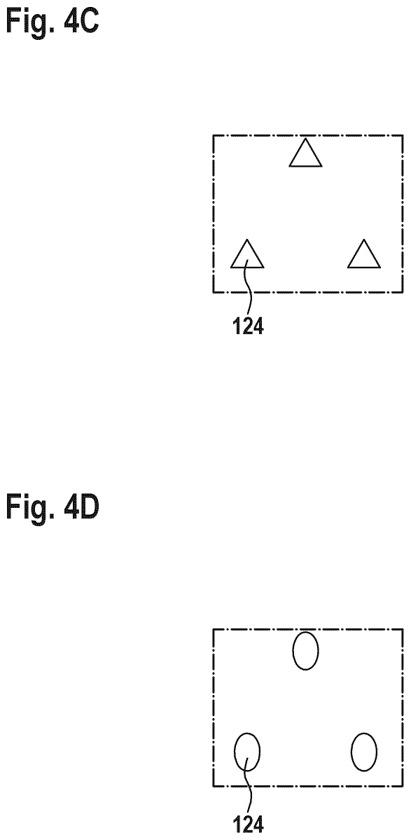

FIG. 4A shows a top plan view of the first frame component base body 10 with the first supporting component 12. Multiple structures 124 are formed in the trapezoidal protrusion 122 that abut or penetrate the supporting component 12. The structures 124 serve to distribute the fluid introduced via the cannula connector 33, of which the cannula connector half 103 is shown here. Elongated fluidic barriers 125 are formed at the edges of the trapezoidal protrusion 122 that additionally prevent leakage of the liquid.

FIG. 4B shows a top plan view of the excerpt B of FIG. 4A, in which a cannula connector half 103 is shown. Here as well, the cannula connector half 103 comprises a widening 330 in the upper region as an injection aid, in the middle region a narrower cannula channel, also referred to as constriction 331, and in the lower region a cavity 332. Reference is made to the description for FIG. 3B.

FIGS. 4C and 4D show two different exemplary embodiments for the structures 124 for dispersing the fluid. In FIG. 4C, the structures 124 have a triangular shape, with a tip pointing towards the cannula connector 33 or the cannula connector half 103, respectively. In FIG. 4D, the structures 124 are round. They may be circular or oval as shown here. The structures 124 provide obstacles to the fluid flow so that it is deflected and thus distributed.

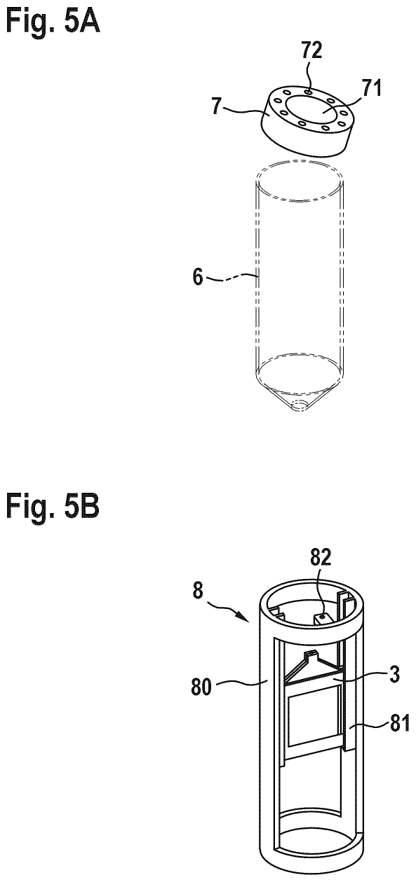

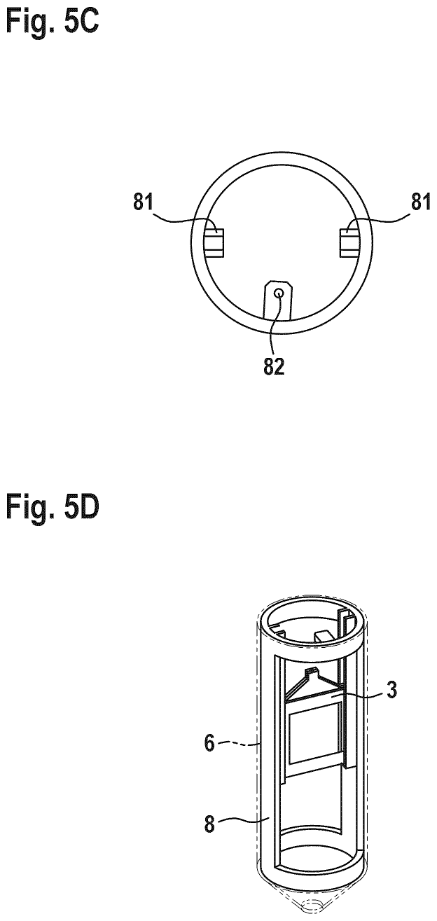

In FIG. 5A, a tube 6 is shown, which serves as a receiving vessel for the device according to the invention and in which the culturing of the tissue section can take place. The tube is cylindrical and has a tapered tip at the bottom end. The tube 6 has a lid 7, which can be placed on the tube 6 in a fluid-tight manner. The lid 7 has a septum 71 on the top side and a plurality of filter openings 72 around the outside. FIGS. 5B and 5C show a retaining frame 8, which can receive and hold the device according to the invention. The retaining frame 8 has an elongated cylindrical frame structure 80, which is adapted in shape and size to the tube 6. Two guide rails 81, which face inwardly on the frame structure 80, are arranged from the upper edge to the center of the frame structure 80. The frame 3 of the device, including the supporting components 12, 22 and the fabric section 4, is inserted into the guide rails 81 from above and then held centrally in the frame structure 80 by the guide rails 81. The frame 3 is inserted so that the cannula connector 33 faces upwards. In addition, the retaining frame has a retaining and guide structure 82 for a cannula formed by an opening in a protrusion at the top of the frame structure 80. In FIG. 5D, the retaining frame 8 is inserted into the tube 6. The frame structure 80 abuts the inner wall of the tube 6 and stands on the tapered tip. The frame 3 of the device according to the invention is held centrally in the tube 6.

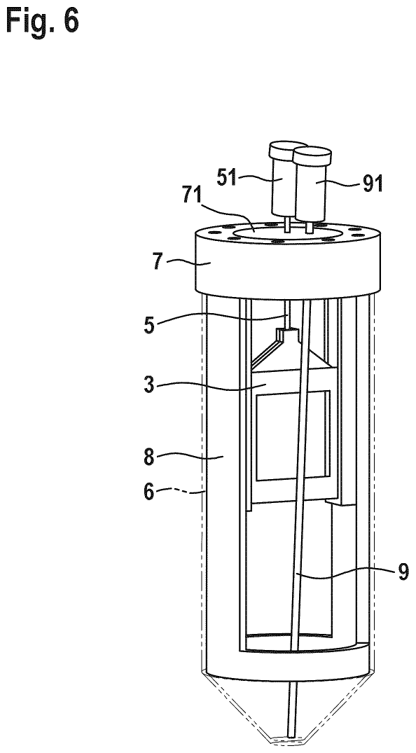

FIG. 6 shows the system according to the invention comprising the device according to the invention having the frame 3 and the supporting components 12, 22, the tube 6, the lid 7, the retaining frame 8, the injection cannula 5 and a removal cannula 9. The device according to the invention is pushed into the retaining frame 8 and the retaining frame is in turn inserted into the tube 6 as shown in FIG. 5D and described in this context. The lid 7 is placed on the tube 6 and seals it in a fluid-tight manner. The injection cannula 5 and the removal cannula 9 are guided through the septum 71 in the lid. On the outer side, the cannulas 5, 9 each have a Luer connector 51, 91 with which the cannulas can be connected to a supply and/or a discharge system. The injection cannula 5 is guided into the cannula connector 33 of the frame 3. Fluid can be introduced into the cannula connector 33 within the frame 3 via the injection cannula 5 and finally into the chamber between the supporting components 12, 22. The removal cannula 9 is guided through the holding and guiding structure 82 of the retaining frame 8 and runs past the frame 3 to the tip of the tube 6. Fluid which has escaped through the supporting components 12, 22 from the device may be removed from the tube 3 through the removal cannula 9.

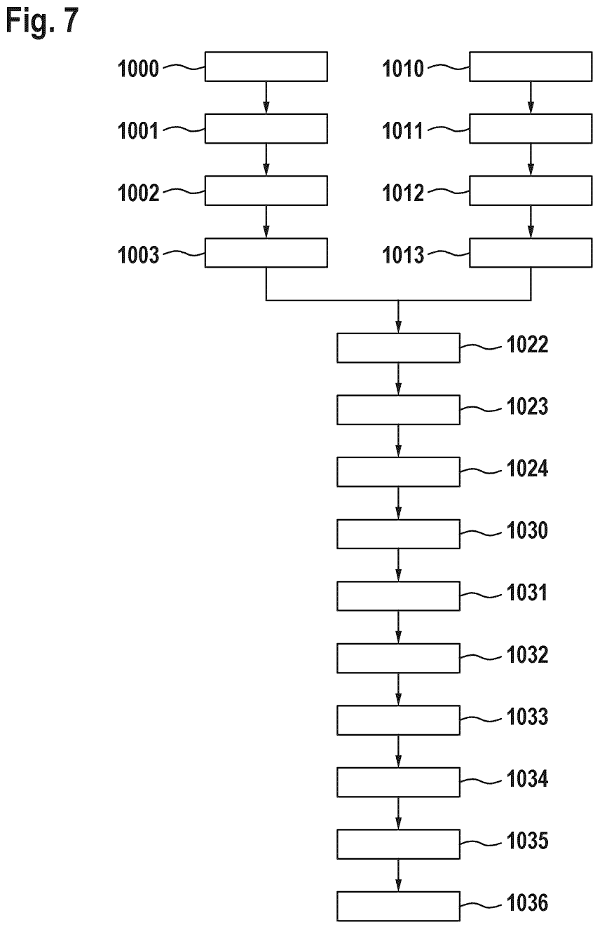

FIG. 7 shows a flowchart of an exemplary embodiment of the method for culturing a tissue section 4 according to the invention. With reference to FIGS. 1A-D, a first frame component base body 10 and a first frame component insert 11 are provided 1000, a first supporting component 12 is placed 1001 on the first frame component insert 11, the first frame component base body 10 is placed 1002 on the first frame component insert 11, wherein the collar 112 of the first frame component-insert profile 110 is pushed into the opening 101 of the first frame component base body 10, and the first supporting component 12 is passed through 1003 the opening 101 of the first frame component base body 10 on the side of the trapezoidal protrusion 102 as well as on the opposite side, such that a first frame component 11 is formed. Accordingly, with reference to FIGS. 1E-H, a second frame component 12 is formed, in that a second frame component base body 20 and a second frame component insert 21 are provided 1010, a second supporting component 22 is placed on top of 1011 the second frame component insert 21, the second frame component base body 20 is placed on top of 1012 the second frame component insert 21, wherein the collar 212 of the second frame component insert profile 210 is pushed into the opening 201 of the second frame component base body 20, and the second supporting component 22 is passed through 1013 the side of the trapezoidal protrusion 202 as well as on the opposite side through the opening 201 of the second frame component base body 20.

The fabric section 4 is then placed 1022 on one of the supporting components 12, 22, for example the second supporting component 22, in the second frame component 2. The first frame component 1 is then placed 1023 on the second frame component 2. The two frame components 1, 2 are then connected to each other 1023 and thus the frame 3 of the device according to the invention, as shown in FIG. 2B, is produced. The fabric section 4 is placed between the supporting components 12, 22. The connecting elements 214 on the second frame component insert 21 are clipped into the first frame component insert 11 to create a rigid, yet releasable, connection of the frame components 1, 2. The frame 3 with the supporting components 12, 22 and the tissue section 4 is then pushed 1030 into a retaining frame 8 and the retaining frame 8 as well as the frame 3 with the supporting components 12, 22 and the tissue section 4 are inserted 1031 into a tube 6 as shown in FIG. 5. Then, the tube 6 is closed 1032 with a lid 7 and placed 1033 in an incubator (not shown). The device according to the invention is then fluidly contacted 1034 in that, as shown in FIG. 6, the injection cannula 5 is inserted through the septum 71 in the lid 7 into the cannula connector 33 of the frame 3 and is fluidly connected to a supply system via the Luer connector 51, and the removal cannula 9 is also passed through the septum 71 in the lid 7 to the tip of the tube 6 and is fluidly connected to a collection system via the Luer connector 91. Finally, a culturing medium with a pre-determinable composition of nutrients and a pre-determinable oxygen concentration is fluidly supplied 1035 via the supply system and the injection cannula 5 over a pre-determinable period of time. The culturing medium that has leaked through the supporting components and come into contact with the tissue section 4 is then removed 1036 via the removal cannula 9.

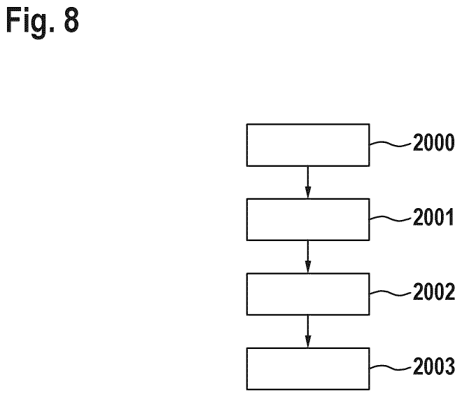

FIG. 8 shows a flowchart of a method for medication testing. Via the supply system and the injection cannula 5, a medication solution with a pre-determinable dose and concentration is supplied 2000 fluidly to the device according to the invention and thus to the tissue section 4 over a pre-determinable period of time. The supply of the culturing medium can be maintained as described in FIG. 7 in step 1035. The fluid recirculated in step 1036 is fed to an analysis system and examined 2001 therein. Steps 2000 and 2001 may be repeated with various medication solutions. Finally, the fluid feed is stopped 2002, i.e., both the supply of the culturing medium and the supply of medication solution. The tissue section 4 is finally removed by removing the retaining frame 8 from the tube 6, pulling the frame 3 out of the retaining frame 8, and separating the two frame components 1, 2 by disconnecting 2003 the connecting elements 214. The frame components 1, 2 can be disassembled by detaching the frame component insert 11 or 21 from the frame component base body 10 or 20, respectively. Then, the supporting components 12, 22 can be removed and also examined.

1. A device for culturing a tissue section comprising:
a frame having a circumferential profile and a central opening defined by the circumferential profile, the frame comprising two frame components, each of which has a circumferential component profile and a respective central component opening defined by the circumferential component profile, which the two frame components being configured to be fitted together flush with one another to form the frame; and
two supporting components wherein the two supporting components are configured to receive a tissue section,
wherein each frame component of the two frame components comprises a frame component base body and a frame component insert, which are configured to be assembled to form the frame component,
wherein for each frame component, the frame component insert and the frame component base body are configured to hold the supporting component such that the supporting component is clamped on the frame component spanning the respective central component opening.

2. The device according to claim 1, wherein a connecting element is provided by means of which the frame components can be assembled to the frame, wherein the connecting element is configured on a frame component insert, and is connectable to the other frame component insert.

3. The device according to claim 2, wherein the connecting element is configured as a click connection, a guide rail, a clamp connection or a fastener.

4. The device according to claim 1, wherein the frame and/or the two frame components comprise a guide for supplying fluid.

5. The device according to claim 4, wherein the guide is configured for supplying fluid in the assembled frame component base bodies.

6. The device according to claim 4, wherein the guide for supplying fluid within the frame profile has a widening in an outer section, has a constriction in a central section, and forms a cavity in an inner section.

7. The device according to claim 1, wherein:
the frame component base body has a trapezoidal protrusion on one side which widens towards the component opening,
the supporting component has a trapezoidal side, and
the trapezoidal side of the supporting component rests on the frame component base body on a side facing away from the frame component insert.

8. The device according to claim 7, wherein the frame component base body has structures on a surface of the trapezoidal protrusion for distributing the fluid, and the structures abut or penetrate the supporting component.

9. A culturing system for culturing tissue sections, comprising:
the device according to claim 1; and
a receiving vessel, wherein the receiving vessel comprises a retaining frame holding the device in the receiving vessel.

10. The culturing system according to claim 9, wherein the receiving vessel is a ventable tube.

11. The culturing system according claim 9, wherein the receiving vessel comprises a lid through which an injection cannula and/or a removal cannula is guided.

12. A method for culturing a tissue section with the device according to claim 1, the method comprising:
preparing each of the two frame components by placing the supporting component on the frame component insert for each frame component, the supporting component spanning the component opening and extending into the profile of the frame component insert, the frame component insert being inserted into the frame component base body so that the supporting component is held;
placing a fabric section on a supporting component in a frame component;
placing the other frame component on top, wherein the two frame component base bodies lie on top of each other, the supporting component of the other frame component rests on the fabric section;
connecting the frame components to a frame;
supplying fluids in the opening surrounded by the supporting components within the frame.

13. The method according to claim 12, wherein the two frame components are connected by connecting a connecting element formed on the frame component insert of one of the two frame components to the frame component insert of the other frame component of the two frame components.

14. The method according to claim 12, wherein the frame is pushed into a retaining frame, and the frame and the retaining frame are inserted together into the receiving vessel.

15. The method according to claim 14, further comprising:
closing the receiving vessel with a lid;
guiding an injection cannula to the frame through the lid for the supply of fluids; and
guiding a removal cannula into the receiving vessel for the removal of fluids through the lid.
